(19) United States
(12) Patent Application Publication
(10) Pub. No.: US 2023/0413927 A1
(43) Pub. Date: Dec. 28, 2023

(54) PROTECTIVE TOE COVERING

(71) Applicant: Cia-Mattae Saunsiderae Sevier, Chicago, IL (US)

(72) Inventor: Cia-Mattae Saunsiderae Sevier, Chicago, IL (US)

(21) Appl. No.: 18/343,194

(22) Filed: Jun. 28, 2023

Related U.S. Application Data
(60) Provisional application No. 63/356,200, filed on Jun. 28, 2022.

(51) Int. Cl. A41D 13/06

(52) U.S. Cl. CPC A41D 13/06

(57) ABSTRACT

A protective toe covering, associated kit, and method of application for the protective toe covering to a toe of a wearer is disclosed. The protective toe covering includes a protective cover with adhesive that allows the protective toe covering to adhere to skin. The associated protective toe covering kit includes a case with a lid pivotally coupled to a base. The base provides spaces to loosely hold sheets of protective toe coverings and a pick-up tool. The method of application of the protective toe covering includes a wearer opening the lid of the case, selecting the pick-up tool and a sheet of protective toe coverings, selecting a desired protective toe covering using the pick-up tool, and causing the protective toe covering to adhere to a desired toe using the pick-up tool.

### CROSS-REFERENCE TO RELATED APPLICATION

This application claims the benefit of priority of U.S. provisional application No. 63/356,200, filed Jun. 28, 2022, the contents of which are herein incorporated by reference.

### BACKGROUND OF THE INVENTION

The present invention relates to foot accessories and, more particularly, to a protective toe covering.

Many people develop callouses or open sores on their feet and toes throughout their life. The constant movement and pressure on feet, from simply standing, walking, or wearing certain pairs of shoes, provide the perfect environment for callouses and sores to develop. When these sores or callouses develop, it can be painful and difficult for the affected person to walk or otherwise move on their feet. Further, the open sores and callouses can be unsightly and increase the risk of infection for the affected person.

Conventional solutions for protecting toes and feet from callouses or open sores involve bulky devices that are inconvenient to use with conventional footwear, and often are provided in boring or ugly colors. Further, some conventional solutions include medication that may be unnecessary and that may produce unwanted side effects.

As can be seen, there is a need for a non-bulky device that provides aesthetically pleasing and comfortable protection for toes and feet against open sores and callouses. The present invention solves these issues by providing a protective toe covering that provides multiple options for size, color, and shape of the protective covering. The present invention also provides a secure and unobtrusive protective toe covering that allows a wearer to move about comfortably, no matter their choice of footwear.

### SUMMARY OF THE INVENTION

In one aspect of the present invention, a protective toe covering is provided, the protective toe covering comprising a protective cover, having a front face and a rear face, with an adhesive placed on the rear face of the protective cover, wherein the adhesive is configured to adhere to bare skin. The protective cover may comprise medical grade silicone, and the adhesive backing may be latex free. Further, the protective cover may be substantially circular, be provided in a plurality of aesthetically pleasing colors, and be provided in a variety of sizes, including having a diameter of at least one of about 10 mm or about 5 mm.

In another aspect of the present invention, a protective toe covering kit is provided, with the protective toe covering kit comprising a case including a lid pivotally coupled to a base, wherein the base includes a plurality of protective covering compartments and at least one pick-up tool compartment. The protective toe covering kit further comprises a plurality of sheets configured to be loosely retained within a respective one of the plurality of protective covering compartments, with each of the plurality of sheets retaining a plurality of protective toe coverings. Each of the plurality of protective toe coverings comprises a protective cover and an adhesive, the protective cover comprising a front face and a rear face, and the adhesive being placed on the rear face of the protective cover and configured to adhere to bare skin. Additionally, the protective toe covering kit comprises at least one pick-up tool configured to be loosely retained within the at least one pick-up tool compartment.

In another aspect of the present invention, each of the plurality of sheets of the protective toe covering kit comprises a release liner coupled to a silicone sheet in which the plurality of protective toe coverings are formed. Further, the adhesive of each of the plurality of protective toe coverings releasably adheres to the release liner and the adhesive remains only on a respective one of the plurality of protective toe coverings when the respective one of the plurality of toe coverings is removed from the release liner.

In another aspect of the present invention, the pick-up tool of the protective toe covering kit comprises two pincer levers coupled together at a fixed end and each of the two pincer levers having a free end. Additionally, the free end of each of the two pincer levers comprises an elliptical tooth that is integrally connected to the two pincer levers via a first pincer portion and a second pincer portion, with the first pincer portion and the second pincer portion arranged to form an acute angle relative to one another.

In another aspect of the present invention, a method of protecting toes and feet from open sores and callouses is provided. The method comprises the steps of opening a lid of a case, the case further comprising a base including a plurality of protective covering compartments and at least one pick-up tool compartment; grabbing a pick-up tool from within the at least on pick-up tool compartment, and grabbing a sheet comprising a plurality of protective toe coverings from one of the plurality of plurality of protective covering compartments; removing one of the plurality of protective toe coverings from the sheet using the pick-up tool; and adhering the one of the plurality of protective toe coverings to a toe of a wearer using the pick-up tool.

These and other features, aspects and advantages of the present invention will become better understood with reference to the following drawings, description, and claims.

### BRIEF DESCRIPTION OF THE DRAWINGS

FIG. 1 is a top perspective view of a protective toe covering kit in accordance with an embodiment of the present invention;

FIG. 2 is a detailed perspective view of the protective toe covering kit in use in accordance with an embodiment of the present invention; and

FIG. 3 is a top perspective view of a protective toe covering applied to toes in accordance with an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a protective toe covering comprising a protective cover, having a front face and a rear face, with an adhesive placed on the rear face of the protective cover, wherein the adhesive is configured to adhere to bare skin.

Another embodiment of the present invention includes a protective toe covering kit comprising a case having a lid pivotally coupled to a base, wherein the base includes a plurality of protective covering compartments and at least one pick-up tool compartment. The protective toe covering kit further comprises a plurality of sheets configured to be loosely retained within a respective one of the plurality of protective covering compartments, with each of the plurality of sheets retaining a plurality of protective toe coverings. Each of the plurality of protective toe coverings comprises a protective cover and an adhesive, the protective cover comprising a front face and a rear face, and the adhesive being placed on the rear face of the protective cover and configured to adhere to bare skin. Additionally, the protective toe covering kit comprises at least one pick-up tool configured to be loosely retained within the at least one pick-up tool compartment.

An additional embodiment of the present invention provides a method of protecting toes and feet from open sores and callouses is provided. The method comprises the steps of opening a lid of a case, the case further comprising a base including a plurality of protective covering compartments and at least one pick-up tool compartment; grabbing a pick-up tool from within the at least on pick-up tool compartment, and grabbing a sheet comprising a plurality of protective toe coverings from one of the plurality of plurality of protective covering compartments; removing one of the plurality of protective toe coverings from the sheet using the pick-up tool; and adhering the one of the plurality of protective toe coverings to a toe of a wearer using the pick-up tool.

The present invention is thus able to provide a protective toe covering that provides multiple options for size, color, and shape of the protective covering. The present invention also provides a secure and unobtrusive protective toe covering that allows a wearer to move about comfortably, no matter their choice of footwear. The present invention protects the wearer's toes from any breakdown of skin or the creation of open wounds and provides those wearers who have sores or callouses with an improved look for their feet.

It should be noted that the materials of manufacture for the present invention are not particularly limited. For example, the pick-up tool may be formed of plastics, metals, or other similarly hard, durable, and flexible materials.

It should also be noted that the method of manufacture for the present invention is not particularly limited. For example, the pick-up tool may be formed via molding, machining, 3-D printing, or other conventionally known means.

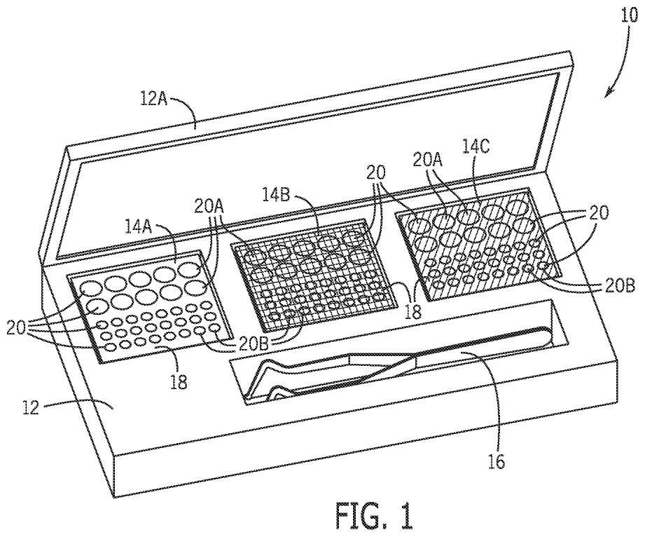

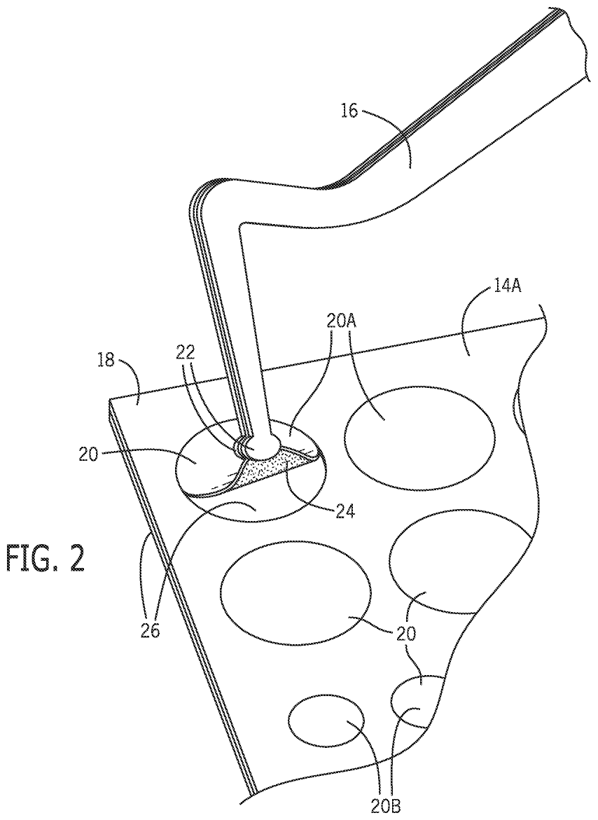

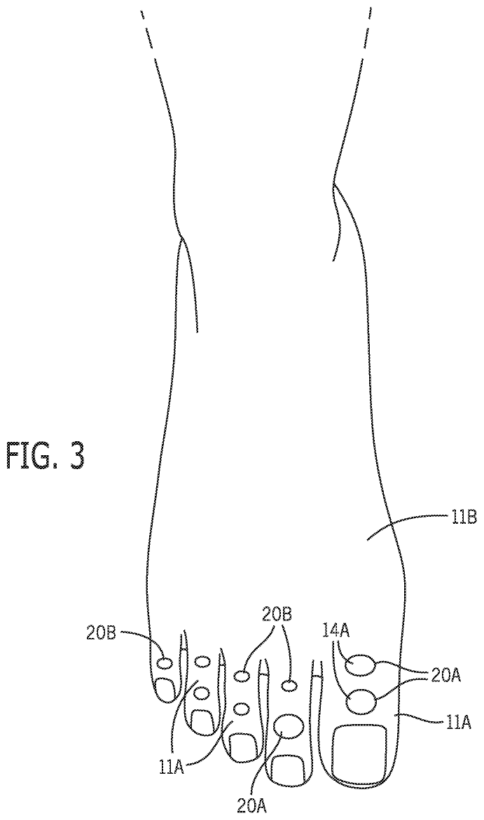

Referring to FIGS. 1-3, an embodiment of a protective toe covering, associated protective toe covering kit, and method of application in accordance with the present invention are shown.

As shown in FIG. 1, a protective toe covering kit 10 includes a case 12 having a base and a lid 12A pivotally coupled to the base. The base includes a plurality of protective covering compartments and at least one pick-up tool compartment. A plurality of sheets 14A, 14B, 14C comprised of medical grade silicone are loosely retained within a respective one of the plurality of protective covering compartments. Each of the plurality of sheets 14A, 14B, 14C is formed of a release liner 26 coupled to a silicone sheet 18 via an adhesive 24. Within each of the plurality of sheets 14A, 14B, 14C, a plurality of protective toe coverings 20, 20A, 20B, may be cut out, stamped out or otherwise formed within and retained within the respective silicone sheet 18 and adhesive 24. Each of the plurality of sheets 14A, 14B, 14C, may include at least two different sizes of protective toe coverings 20, 20A, 20B. For example, a large sized protective toe covering 20A may have a diameter of about 10 mm, while a small sized protective toe covering 20B may have a diameter of about 5 mm.

Each of the plurality of protective toe coverings 20, 20A, 20B comprises a protective cover 20 with a front uncovered face and a rear face with adhesive 24 applied thereto. The protective cover 20 may comprise medical grade silicone, with the adhesive 24 being configured to adhere to bare skin. The adhesive 24 may additionally be latex free to enable people with latex allergies to use the present invention. The protective cover 20 is releasably adhered to the release liner 26 via the adhesive 24. When one of the protective covers 20 is removed from a respective one of the plurality of sheets 14A, 14B, 14C, the adhesive 24 is removed with the protective cover 20 such that no adhesive 24 remains on the portion of the release liner 26 where the protective cover 20 was removed.

The protective cover 20 may be substantially circular, and may be provided to a wearer in a plurality of different aesthetically that match the respective one of the plurality of sheets 14A, 14B, 14C, in with they are retained.

Additionally, the protective toe covering kit comprises at least one pick-up tool 16 configured to be loosely retained within the at least one pick-up tool compartment. The pick-up tool 16 comprises two pincer levers coupled together at a fixed end and each of the two pincer levers has a free end. Each free end is movable between a resting position and a compressed position. In the resting position, the free ends are distal from one another, and in the compressed position, the free ends may be made to be proximal to one another. Additionally, the free end of each of the two pincer levers comprises a blunt elliptical tooth 22 that is integrally connected to the two pincer levers via a first pincer portion and a second pincer portion, with the first pincer portion and the second pincer portion arranged to form an acute angle relative to one another. The second pincer portion may include a curvature that forms an obtuse angle in an opposite direction relative to the acute angle formed between the first pincer portion and second pincer portion.

In operation, a wearer may open the lid 12A of the case 12 and utilized the protective toe cover kit after first washing and drying a portion of the wearer's toes 11A and feet 11B to create a clean and oil-free skin surface on which the wearer intends to apply the protective toe coverings 20, 20A, 20B. The wearer may then grab the pick-up tool 16 from within the at least on pick-up tool compartment of the case 12, and grab a desired one of the plurality of sheets 14A, 14B, 14C, from one of the plurality of protective covering compartments, depending on the wearer's aesthetic preferences. The wearer may then use the pick-up tool 16 to select a desired one of the protective toe coverings 20, 20A, 20B, and thereby preventing oil and/or dirt from being transferred to the adhesive 24. To do so, the wearer may place the blunt elliptical teeth 22 of the free ends of the pick-up tool 16 adjacent an edge of one of the plurality of protective toe covers 20, 20A, 20B. Upon compressing the pick-up tool 16, the wearer may peel the desired protective toe cover 20, 20A, 20B and associated adhesive 24 from the release liner 26 and surrounding silicone sheet 18. Once peeled, the wearer may place and cause the protective toe cover 20, 20A, 20B, to adhere on the desired portion of the wearer's toes 11A or feet 11B. By doing this, the wearer is able to protect their toes 11A and feet 11B from developing open sores or callouses, or protect any previously developed sores or callouses from further irritation or damage.

The present invention thus provides a seamless and aesthetically pleasing means and method for protecting a wearer's toes and feet, no matter their age, gender, or choice of footwear. The present invention is unobtrusive, provides protection over extended periods of time, and easily allows a wearer to use their desired footwear during those times.

It should be understood, of course, that the foregoing relates to exemplary embodiments of the invention and that modifications may be made without departing from the spirit and scope of the invention as set forth in the following claims.

What is claimed is:

1. A protective toe covering comprising:
a protective cover, comprising a front face and a rear face;
an adhesive placed on the rear face of the protective cover, wherein the adhesive is configured to adhere to bare skin.

2. The protective toe covering of claim 1, wherein the protective cover comprises medical grade silicone.

3. The protective toe covering of claim 1, wherein the adhesive backing is latex free.

4. The protective toe covering of claim 1, wherein the protective cover is substantially circular and has a diameter of at least one of about 10 mm or about 5 mm.

5. A protective toe covering kit comprising:
a case comprising a lid pivotally coupled to a base, wherein the base includes a plurality of protective covering compartments and at least one pick-up tool compartment;
a plurality of sheets configured to be loosely retained within a respective one of the plurality of protective covering compartments, each of the plurality of sheets retaining a plurality of protective toe coverings;
wherein each of the plurality of protective toe coverings comprises a protective cover and an adhesive, the protective cover comprising a front face and a rear face, and the adhesive being placed on the rear face of the protective cover and configured to adhere to bare skin; and
at least one pick-up tool configured to be loosely retained within the at least one pick-up tool compartment.

6. The protective toe covering kit of claim 5, wherein each of the plurality of sheets comprises a release liner coupled to a silicone sheet in which the plurality of protective toe coverings are formed.

7. The protective toe covering kit of claim 6, wherein the adhesive of each of the plurality of protective toe coverings releasably adheres to the release liner and the adhesive remains only on a respective one of the plurality of protective toe coverings when the respective one of the plurality of toe coverings is removed from the release liner.

8. The protective toe covering kit of claim 5, wherein the pick-up tool comprises two pincer levers coupled together at a fixed end and each of the two pincer levers having a free end.

9. The protective toe covering kit of claim 8, wherein the free end of each of the two pincer levers comprises an elliptical tooth that is integrally connected to the two pincer levers via a first pincer portion and a second pincer portion, with the first pincer portion and the second pincer portion arranged to form an acute angle relative to one another.

10. A method of protecting toes and feet from open sores and callouses, the method comprising the steps of:
opening a lid of a case, the case further comprising a base including a plurality of protective covering compartments and at least one pick-up tool compartment;
selecting a pick-up tool from within the at least on pick-up tool compartment;
selecting a sheet comprising a plurality of protective toe coverings from one of the plurality of plurality of protective covering compartments;
removing one of the plurality of protective toe coverings from the sheet using the pick-up tool; and
adhering the one of the plurality of protective toe coverings to a toe of a wearer using the pick-up tool.
